(19) United States
(12) Patent Application Publication
(10) Pub. No.: US 2018/0037650 A1
(43) Pub. Date: Feb. 8, 2018

(54) Pain Treatment

(71) Applicant: Biosceptre (UK) Limited, Cambridgeshire (GB)

(72) Inventor: Julian Alexander Barden, North Ryde (AU)

(21) Appl. No.: 15/555,959

(22) PCT Filed: Apr. 1, 2016
(86) PCT No.: PCT/AU2016/050249

§ 371 Date: Sep. 5, 2017

(30) Foreign Application Priority Data
Apr. 2, 2015 (AU) 2015901215
Apr. 2, 2015 (AU) 2015901216

(51) Int. Cl. C07K 16/28; A61K 39/395; A61K 38/17; C07K 16/42

(52) U.S. Cl. CPC C07K 16/28; C07K 16/42; A61K 39/395; A61K 38/177; A61K 2039/505

(57) ABSTRACT

A method of managing pain in an individual including the following steps: providing an individual in need of pain management; administering an anti-P2X₇ receptor antibody to the individual; thereby managing pain in the individual.

### FIELD OF THE INVENTION

The invention relates to the treatment of chronic pain, in particular to the treatment of cancer pain.

### BACKGROUND OF THE INVENTION

Reference to any prior art in the specification is not an acknowledgment or suggestion that this prior art forms part of the common general knowledge in any jurisdiction.

Chronic pain is pain that generally extends beyond the expected period of healing. Some definitions of chronic pain refer to pain that has lasted longer than 3 to 6 months from inception, as compared with acute pain, which may last less than 30 days and subacute pain of 1 to 6 months duration. Where a relevantly associated condition is untreatable or does not heal, chronic pain may exist for the duration of the condition. Thus, chronic pain may be commonly observed in many forms of cancer and diabetes.

Chronic pain may be in the form of ‘nociceptive’ pain arising from persistent activation of nociceptors. In some circumstances, the pain may arise from, or be facilitated by the generation of non-nociceptive nerve fibres that generate and respond to pain signals. ‘Superficial nociceptive pain’ is activated by nociceptors in the skin or superficial tissues, whereas ‘deep nociceptive pain’ may originate from organs (i.e. ‘visceral pain’) or from somatic tissue (‘somatic pain’) such as ligaments, tendons, bones, blood vessels, fasciae and muscles. Somatic pain may present as aching, poorly-localised pain, whereas visceral pain, while well localised, may be referred pain, and therefore it may be difficult to determine the source of the pain.

Chronic pain may also present in the form of ‘neuropathic’ pain, which arises from injury to, or degeneration, of the nervous system. Nervous system injury may arise in various cancers, (for example from nervous tissue compression or invasion in cancer), diabetes, infection, autoimmune disease and traumatic or physical injury to the peripheral or central nervous tissue. ‘Peripheral neuropathic pain’ originates from the peripheral nervous system and may present in the form of burning, tingling, stabbing or pins and needle sensations. ‘Central neuropathic pain’ arises from the brain or spinal cord.

‘Tactile allodynia’, which is a pain hypersensitivity to normally innocuous stimuli may be observed in individuals suffering from chronic pain. ‘Hyperalgesia’, which is an increased pain perception of noxious stimuli may be another symptom of chronic pain.

Chronic pain in the form of cancer pain may be mechanistically classified as nociceptive or neuropathic pain. Nociceptive pain most commonly occurs from pressure, infiltration or tumor invasion into structures with high sensitivity to noxious stimulation such as bones, viscera, pleura, blood vessels and other soft tissues. Pain from metastatic tumors that damages bones, from involvement of pleural membranes, distension of capsular organs, pleural effusion and ascites are common. Nerve infiltration or invasion by malignant or metastatic tumours, or nerve compression from growth of benign tumours, may be a particular cause of neuropathic pain.

The involvement of ATP and purinergic signalling in nociception has long been recognised. In 1966 it was demonstrated that ATP could initiate pain when applied to human skin. In the 1990s, extracellular ATP was accepted as a functional neurotransmitter Falk S. et al. 2012 J. Osteo. Article ID 758181. Today the role of ATP in pain transmission is well established: Tsuda M. et al. 2010 Brain Res. Rev. 63: 222-232.

Tumour sites are known to be associated with increased levels of extra cellular ATP and in the tumour interstitium, ATP is present in the hundreds micromolar range, while basically undetectable in healthy tissue. (Pellegatti P, Raffaghello L, Bianchi G, Piccardi F, Pistoia V, et al. (2008) Increased Level of Extracellular ATP at Tumor Sites: In Vivo Imaging with Plasma Membrane Luciferase. PLoS ONE 3(7): e2599. doi:10.1371/iournal.pone.0002599). It is believed that these tumour localised high concentrations may be the origins of cancer-associated pain signals.

A range of nociceptors on sensory nerve fibres are believed to be involved in transmission of an ATP-mediated pain signal. In particular, ATP-mediated agonism of purinergic receptors, including the P2X ligand-gated ion channel receptors and the P2Y G protein-coupled receptors, is now established as being directly involved in pain transmission. These receptors have a key role in the development of neuropathic pain and in nociceptive pain transmission (Falk et al. supra).

A crucial role has been proposed for receptors containing P2X₃ subunits in mediating the primary sensory effectors of ATP. The relevant receptors are predominantly localized to the small to medium diameter C- and Aδ-fibre sensory neurons within the dorsal root ganglion and cranial sensory ganglia and on their peripheral nerve terminals in receptive fields in tissues including the skin, joints and viscera. P2X₃ subunits are also present on the central projections of these primary sensory neurons within the dorsal horn of the spinal cord and in the brain stem (Ford A., 2012 Purinergic Signalling 8 (Suppl 1): S3-S26; Chen, Y, et al 2012 Molecular Pain 8:9)).

P2X₇ receptors have also been identified as having some involvement in ATP-mediated pain signal transmission in some animal models, although the extent to which they are involved in transmission of pain signals is unknown given the significantly lower expression of these receptors on nerve tissue, as compared with other purinergic receptors such as P2X₁ receptors (See Hughes J P et al. 2007 Purinergic Signaling 3:163-169). These receptors consist of two transmembrane domains with intracellular N- and C-terminals and a long extracellular loop between the transmembrane regions. The extracellular domain contains ATP binding sites for ATP, competitive antagonists and modulatory metal ions. Falk S. et al. 2012 J. Osteo. Article ID 758181. A characteristic of the P2X₇ receptor, but also some of the other P2X receptors is the ability to induce pore formation allowing the permeation of large molecules.

Studies using transgenic KO mice and specific receptor antagonists have shown a role for P2X₇ receptors in mediating chronic pain in animal models, although the underlying mechanisms are not understood. It is believed that the P2X₇ pore, which is formed on binding of ATP to the P2X₇ receptor is critical for determining the sensitivity to chronic pain (Jian L. et al. 2013 Frontiers in Pharmacology Vol. 4, Article 55 p 1-17). More specifically, individuals who have impaired pore function, and individuals with normal pore function who had been treated with peptide to occlude the pore, were seen to have reduced allodynia (Sorge R. et al. 2013 Nat. Med. 18(4):595-599). In other studies, a P2X₇ receptor antagonist, A839977 was found to be analgesic in a model of cancer induced bone pain Falk S et al. 2015 Neuroscience S0306-4522. Further, lidocaine, a widely used analgesic has recently been shown to inhibit ATP induced currents across P2X₇ receptors (Okura D. et al. 2015 Anesth. Analg. 120:597-605), again suggesting antagonism of ATP-induced P2X₇ receptor function enables analgesia.

One particular variant of the P2X₇ receptor is known to have some deficiency in ATP binding, one consequence of which is that the receptor has limited capacity to form a pore for the ingress of large molecules. This receptor may otherwise permit the ingress or egress of smaller compounds. This receptor has been variously referred to as a ‘non functional’ ‘P2X₇ receptor, reflecting the limited capacity of the receptor for binding ATP. This receptor variant is not involved in ATP-mediated pain signal transmission because it has limited capacity for ATP binding and it is not found on sensory nerve fibres that transmit pain signals.

Chronic and cancer pain may be refractory to currently available treatments such as non steroidal anti-inflammatory drugs, opioids, analgesics and local anaesthetics. However, the side effects of these treatments are well known, and include the suppression of the perception of acute pain arising from frank tissue injury, leaving the individual partially insensitive to harmful mechanical or thermal injury. As in chronic pain situations, it is the pain itself that has become largely pathological, the focus should be on targeting the mechanisms that lead from a change from normal to abnormal sensory perception, rather than providing a general systemic analgesia.

One approach to the development of new therapeutics has been to target antagonism of the ATP-purinergic receptor interaction. P2X₃ antagonists for chronic pain and afferent sensitisation are of key interest (Ford A., supra). Similar approaches have been proposed for antagonism of P2X₇ and other P2X and P2Y receptors. One potential limitation of the approach is that it relies on maintaining a therapeutically effective amount of antagonist for antagonism of the ATP-receptor interaction. More specifically, as the approach does not minimise the amount of local ATP where activation of nociceptive neurons occurs, chronic pain would return when the amount of antagonist drops below the therapeutic level. Another problem is that as ATP-mediated pain signals can be transmitted by a range of unrelated nociceptors, a wide range of antagonists could be required to minimise pain or otherwise to induce analgesia.

There remains a need for new approaches to the treatment of chronic pain, and in particular for treatment of cancer pain.

There is a need for new approaches to treat symptoms of systemic pain, which may arise from nociceptive pain in the form of visceral or somatic pain.

There is a need to improve the quality of life of individuals, preferably individuals in palliative care having cancer pain by minimising or reducing chronic pain, especially systemic pain.

### SUMMARY OF THE INVENTION

In one embodiment there is provided a method of managing pain in an individual including the following steps:
providing an individual in need of pain management;
administering an anti-P2X₇ receptor antibody to the individual;

thereby managing pain in the individual.

In embodiments the method may further include the step of:
assessing the individual to determine the level of reduction of symptoms of the pain in the individual after administration of the anti-P2X₇ receptor antibody;

thereby managing pain in the individual. Subsequent further administration and assessment steps may be implemented to manage pain in the individual.

In another embodiment there is provided a use of an anti P2X₇ receptor antibody in the manufacture of a medicament for the treatment of pain in an individual.

In another embodiment there is provided an anti P2X₇ receptor antibody for use in the treatment of pain in an individual.

In another embodiment there is provided multimeric antibody for minimising cancer pain including:
at least a first variable domain for binding to a P2X₇ receptor for minimising cancer pain
at least a second variable domain for binding to a cancer cell antigen.

In another embodiment there is provided a pharmaceutical composition including a multimeric antibody as described above.

In one embodiment there is provided a method of managing pain in an individual including the following steps:
providing an individual in need of pain management;
administering an anti-P2X₇ receptor, fragment thereof or peptide derived therefrom to the individual thereby forming an humoral immune response to the receptor, fragment or peptide in the individual;

thereby managing pain in the individual.

In embodiments the method may further include the step of:
assessing the individual to determine the level of reduction of symptoms of the pain in the individual after administration of the anti-P2X₇ receptor, fragment thereof or peptide derived therefrom;

thereby managing pain in the individual. Subsequent further administration and assessment steps may be implemented to manage pain in the individual.

In another embodiment there is provided a use of an anti-P2X₇ receptor, fragment thereof or peptide derived therefrom in the manufacture of a medicament for the treatment of pain in an individual. The anti-P2X₇ receptor, fragment thereof or peptide derived therefrom is utilised to form an humoral immune response to the receptor, fragment or peptide in the individual, thereby managing pain in the individual.

In another embodiment there is provided an anti-P2X₇ receptor, fragment thereof or peptide derived therefrom for use in the treatment of pain in an individual. The anti-P2X₇ receptor, fragment thereof or peptide derived therefrom is utilised to form a humoral immune response to the receptor, fragment or peptide in the individual, thereby managing pain in the individual.

In the above described embodiments relevant to an anti-P2X₇ receptor, fragment thereof or peptide derived therefrom, the receptor, fragment or peptide may be provided in the form of a composition that includes an adjuvant, carrier or hapten.

### BRIEF DESCRIPTION OF THE DRAWINGS

FIG. 1. Assay for production or expression of ATP in tumour cells shows that incubation with increasing concentrations (0 uM (microM), 0.62 uM, 1.25 um and 2.5 uM) of a specific IgG antibody labelled with FITC causes progressive diminution of the total ATP in the culture over a 5 h incubation period (50,000 cells/well in triplicate).

FIG. 2. Confocal microscopy was performed and revealed that half the cells in culture showed a heavy cytoplasmic distribution of the FITC label as shown in the confocal image in FIG. 2. This showed that endocytosis of the labelled antibody at 37 C occurred.

FIG. 3. Another confocal microscopy image showed some COLO205 cells had intense internalisation after 3 h at 37 C.

FIG. 4. A confocal microscopy image of prostate PC3 cells that have endocytosed the specific FITC-labeled anti-nfP2X₇ antibody (type IgG₁) in 2 h. The antibody is seen in the EEA1-labeled endosomes in the cytoplasm beneath the phalloidin-stained actin situated under the plasma membrane. Nuclei are stained blue.

FIG. 5. Similar ATP inhibition was elicited at the same concentrations of two-headed (left hand bars, blue) DID-labelled Ab and single headed domain antibody of type V_{H} (right hand bars, red).

FIG. 6. P2X₇ receptor amino acid sequence.

### DETAILED DESCRIPTION OF THE EMBODIMENTS

P2X receptors consist of two transmembrane domains with intracellular N- and C-terminals and a long extracellular loop between the transmembrane regions. The extracellular domain contains ATP binding sites for ATP, competitive antagonists and modulatory metal ions. The N-terminal has similar length in all subtypes, whereas the C-terminal varies considerably from 30 residues in the P2X₆ receptor to 240 residues in the P2X₇ receptor. A characteristic of the P2X₇ receptor, but also some of the other P2X receptors is the ability to induce pore formation allowing the permeation of large molecules when the receptor has bound ATP.

One particular variant of the P2X₇ receptor is known to have some deficiency in ATP binding, one consequence of which is that the receptor has limited capacity to form a pore for the ingress of large molecules. This receptor may otherwise permit the ingress or egress of smaller compounds. This receptor has been variously referred to as a ‘non functional’ P2X₇ receptor, reflecting the limited capacity of the receptor for binding ATP. The limited capacity for ATP binding is understood to arise from the loss of two or all three of the ATP binding sites that are found on functional receptors (i.e. receptors having 3 ATP binding sites).

The non functional P2X₇ receptor is an established biomarker of cancer. In particular, this receptor is expressed on the surface of most cancer cells whether of mesodermal, ectodermal or endodermal origin, and irrespective of cell ontogeny.

Antibodies may be developed that bind to non functional P2X₇ receptors, but not to functional P2X₇ receptors (i.e. receptors that contain all three ATP binding sites to form the pore).

Given that non functional P2X₇ receptors are not found on the surface of sensory neurons, and the limited ability of the non functional P2X₇ receptor to bind ATP and to form the relevant pore, it was expected that the non functional receptor would have a limited role in pain transmission in cancer, particularly given the reduction in pain transmission known to occur in individuals who are unable to form an ATP-mediated pore, or who have been treated with a peptide that occludes a pore formed on ATP-receptor binding. However, the inventor has found that there is a linkage between ATP-mediated pain transmission and non functional P2X₇ receptors.

In particular, as exemplified herein, the inventor has observed in vitro that cancer cells that have been subjected to antibody-induced endocytosis of P2X₇ receptors are deficient in the synthesis and/or secretion of ATP. Further, in clinical studies described further herein, the inventor has observed that individuals who receive an anti-non functional P2X₇ receptor antibody have decreased chronic pain, to the extent that standard opioid treatments for cancer pain management can be withdrawn with no increase in neuropathic or nociceptive pain.

These observations suggest a relationship between cancer cell surface expression of P2X receptors and ATP synthesis and/or secretion by cancer cells, according to which cancer cells synthesise and/or secrete ATP when P2X receptors are expressed on the cancer cell surface, but not when these receptors are removed from the cancer cell surface. ATP secretion by the cancer cells is effectively inhibited or limited when P2X receptors are removed from the cancer cell surface.

While the molecular mechanism underlying the relationship between P2X expression and ATP synthesis/secretion is not fully understood, it is believed that the mechanism for minimising chronic pain is based on the limited availability of cancer cell-derived purinergic receptor agonist at the site where nociceptive neurons are present. In particular, the reduction in P2X cancer cell surface expression appears to reduce the synthesis and/or release of ATP by cancer cells. This effectively starves the local nociceptive neurons of ATP supply, thereby limiting pain transmission through afferent fibres that would otherwise occur when purinergic receptors (for example P2X₃) on the nociceptive neurons are bound by ATP.

Importantly, the inventor recognises that the mechanism for minimisation of chronic pain does not arise from antibody-induced killing of cancer cells that are effectively a potential reservoir of ATP synthesis and/or release. First, the chronic pain reduction is observed in vivo within days of administration of anti-P2X₇ receptor antibody, and in particular at a time where the tumor load has not been effectively reduced. Second, total ablation of cancer cells would be expected to have the opposite effect—i.e. it should increase temporary ATP driven pain transmission because cancer cell lysis would release large amounts of ATP into the extracellular space for binding to purinergic receptors on neurons. Third, as exemplified herein, the in vitro data clearly shows reduction of cancer cell ATP synthesis and/or release prior to significant cell killing.

It is understood that the minimisation of chronic pain is based on a mechanism that operates independently of an interaction of ATP with P2X₇ receptors. Specifically, as described herein, the non functional P2X₇ receptors have very limited capacity to bind to ATP and limited capacity to form a pore that has been identified as responsible for allodynia and pain transmission. Instead, the operating principle is simply that reduction in cell surface P2X₇ receptor (independent of P2X₇-ATP interaction) results in a local deficit of ATP with concomitant impact of opportunity for ATP binding by purinergic receptors expressed on local nociceptive neurons. In this context, the invention is in clear contrast to other approaches to chronic pain minimisation that focus on competitive inhibition of ATP binding to purinergic receptors. According to the invention, the focus is on the local depletion of ATP.

### A. Definitions

As used herein, except where the context requires otherwise, the term “comprise” and variations of the term, such as “comprising”, “comprises” and “comprised”, are not intended to exclude further additives, components, integers or steps.

For purposes of interpreting this specification, the following definitions will apply and whenever appropriate, terms used in the singular will also include the plural and vice versa. In the event that any definition set forth conflicts with any document incorporated herein by reference, the definition set forth below shall prevail.

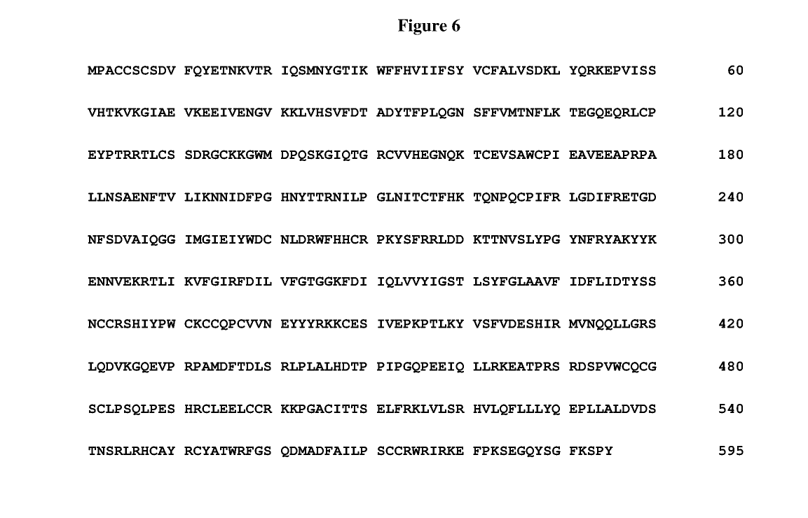

“P2X₇ receptor generally refers to a purinergic receptor formed from three protein subunits or monomers, with at least one of the monomers having an amino acid sequence substantially as shown in SEQ ID NO:1 (see FIG. 6). A P2X₇ receptor may be a functional or non functional receptor as described below. “P2X₇ receptor” encompasses naturally occurring variants of P2X₇ receptor, e.g., wherein the P2X₇ monomers are splice variants, allelic variants and isoforms including naturally-occurring truncated or secreted forms of the monomers forming the P2X₇ receptor (e.g., a form consisting of the extracellular domain sequence or truncated form of it), naturally-occurring variant forms (e.g., alternatively spliced forms) and naturally-occurring allelic variants. In certain embodiments of the invention, the native sequence P2X₇ monomeric polypeptides disclosed herein are mature or full-length native sequence polypeptides comprising the full-length amino acids sequence shown in SEQ ID NO:1. In certain embodiments the P2X₇ receptor may have an amino acid sequence that is modified, for example various of the amino acids in the sequence shown in SEQ ID NO:1 may be substituted, deleted, or a residue may be inserted.

“Functional P2X₇ receptor generally refers to a form of the P2X₇ receptor having a binding site or cleft for binding to ATP. When bound to ATP, the receptor forms a pore-like structure that enables the ingress of calcium ions into the cytosol, one consequence of which may be programmed cell death.

“Non functional P2X₇ receptor generally refers to a form of a P2X₇ receptor in which one or more of the monomers has a cis isomerisation at Pro210 (according to SEQ ID NO:1). The isomerisation may arise from any molecular event that leads to misfolding of the monomer, including for example, mutation of monomer primary sequence or abnormal post translational processing. One consequence of the isomerisation is that the receptor is unable to bind to ATP. In the circumstances, the receptor cannot form a pore and this limits the extent to which calcium ions may enter the cytosol. Non functional P2X₇ receptors are expressed on a wide range of epithelial and haematopoietic cancers.

The term “anti-P2X₇ receptor antibody refers to an antibody that is capable of binding P2X₇ receptor with sufficient affinity such that the antibody is useful as a diagnostic and/or therapeutic agent in targeting P2X₇ receptor, typically non functional P2X₇ receptor. Preferably, the extent of binding of a P2X₇ receptor antibody to an unrelated receptor protein is less than about 10% of the binding of the antibody to P2X₇ receptor as measured, e.g., by a radioimmunoassay (RIA). In certain embodiments, an antibody that binds to P2X₇ receptor has a dissociation constant (Kd) of <1 μM, <100 nM, <10 nM, <1 nM, or <0.1 nM. An anti non functional P2X₇ receptor antibody is generally one having some or all of these serological characteristics and that binds to non functional P2X₇ receptors but not to functional P2X₇ receptors.

“Binding affinity” generally refers to the strength of the sum total of noncovalent interactions between a single binding site of a molecule (e.g., an antibody) and its binding partner (e.g., an antigen). Generally, “binding affinity” refers to intrinsic binding affinity that reflects a 1:1 interaction between members of a binding pair (e.g., antibody and antigen). The affinity of a molecule X for its partner Y can generally be represented by the dissociation constant (K_{d}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

### B. Minimisation and Management of Chronic Pain and Cancer Pain by Adoptive Transfer of Antibody

In another embodiment there is provided a use of an anti P2X₇ receptor antibody in the manufacture of a medicament for the treatment of pain in an individual.

In another embodiment there is provided an anti P2X₇ receptor antibody for use in the treatment of pain in an individual.

In another embodiment there is provided a method of managing pain in an individual including the following steps:
providing an individual in need of pain management;
administering an anti-P2X₇ receptor antibody to the individual;

thereby managing pain in the individual.

In the above described embodiments, the individual may have chronic pain. The chronic pain is localised or systemic.

The pain may be cancer pain. Cancer pain may be in the form of acute or chronic pain, or it may also be “breakthrough” pain. Generally the invention is directed to the treatment of chronic cancer pain.

Cancer pain may be described as nociceptive pain (i.e. somatic pain or visceral pain) and neuropathic pain.

Somatic pain may be deep somatic pain associated with deep tissues such as bone or musculoskeletal elements. It may also be surface or cutaneous somatic pain associated with the dermis or underlying connective tissue. Generally where the pain is somatic pain, it is deep somatic pain, such as bone pain or musculoskeletal pain. Deep somatic pain can be described as dull or aching but localised, whereas cutaneous somatic pain may be sharper or having a pricking or burning sensation. Common causes of deep somatic pain include primary malignant tumour. One example of such a tumour is a sarcoma or osteosarcoma. Another cause may be metastatic disease.

In one embodiment there is provided a use of an anti P2X₇ receptor antibody in the manufacture of a medicament for the treatment of deep somatic cancer pain in an individual arising from a primary tumour being a benign or malignant tumor, or from metastatic disease. Preferably the use results in the reduction of at least one symptom of the pain, preferably a symptom of systemic pain, preferably selected from the group consisting of localised throbbing, dull or aching pain.

In another embodiment there is provided an anti P2X₇ receptor antibody for use in the treatment of deep somatic cancer pain in an individual arising from a primary tumour being a benign or malignant tumor, or from metastatic disease. Preferably the use results in the reduction of at least one symptom of the pain, preferably a symptom of systemic pain, preferably selected from the group consisting of localised throbbing, dull or aching pain.

In another embodiment there is provided a method of managing deep somatic cancer pain in an individual including the following steps:
providing an individual in need of deep somatic cancer pain management arising from a primary tumour being a benign or malignant tumor, or from metastatic disease;
administering an anti-P2X₇ receptor antibody to the individual;

thereby managing pain in the individual. Preferably the method results in the reduction of at least one symptom of the pain, preferably a symptom of systemic pain, preferably selected from the group consisting of localised throbbing, dull or aching pain.

In the treatment of somatic or superficial nociceptive pain, an antibody that binds to functional and non functional P2X₇ receptors, or to non functional P2X₇ receptors only, or to functional P2X₇ receptors only may be used. In one embodiment the antibody binds to functional P2X₇ receptors. In another embodiment the antibody binds to non functional P2X₇ receptors only.

Visceral pain may also be known as soft tissue pain and it refers to pain from a body organ or muscle. Visceral pain may be caused by the activation of pain receptors resulting from infiltration, compression, extension or stretching of the thoracic, abdominal or pelvic viscera. Visceral pain is not well localized and is usually described as pressure-like, deep squeezing. Common causes of visceral pain include pancreatic cancer and abdominal metastases. Other examples of these tumours include liver, gastric or ovarian tumours. Preferably the tumour is a primary malignant tumour in the form of hepatocellular cancer. The tumour may also be metastatic disease arising from colorectal to liver metastases.

In one embodiment there is provided a use of an anti P2X₇ receptor antibody in the manufacture of a medicament for the treatment of visceral cancer pain in an individual arising from a primary tumour being a benign or malignant tumor, or from metastatic disease. Preferably the use results in the reduction of at least one symptom of the pain, preferably a symptom of systemic or referred pain, preferably selected from the group consisting of pain arising from pressure or deep squeezing.

In another embodiment there is provided an anti P2X₇ receptor antibody for use in the treatment of visceral cancer pain in an individual arising from a primary tumour being a benign or malignant tumor, or from metastatic disease. Preferably the use results in the reduction of at least one symptom of the pain, preferably a symptom of systemic or referred pain, preferably selected from the group consisting of pain arising from pressure or deep squeezing.

In another embodiment there is provided a method of managing visceral cancer pain in an individual including the following steps:
providing an individual in need of pain management arising from a primary tumour being a benign or malignant tumor, or from metastatic disease;
administering an anti-P2X₇ receptor antibody to the individual;

thereby managing pain in the individual. Preferably the method results in the reduction of at least one symptom of the pain, preferably a symptom of systemic or referred pain, preferably selected from the group consisting of pain arising from pressure or deep squeezing.

In the treatment of visceral nociceptive pain, an antibody that binds to functional and non functional P2X₇ receptors, or to non functional P2X₇ receptors only, or to functional P2X₇ receptors only may be used. In one embodiment the antibody binds to functional P2X₇ receptors. In another embodiment the antibody binds to non functional P2X₇ receptors only.

In one embodiment, the individual selected for, or provided for treatment is one who has nociceptive pain, especially nociceptive somatic pain, preferably with little or no visceral pain, but not neuropathic pain. In such an individual there may be no significant injury to nerves or nervous tissue.

Neuropathic pain, or nerve pain is caused by injury to the nervous system for example by a tumour compressing nerves or infiltrating and invading nervous tissue. This manifests as burning, shooting or tingling pain. Primary or metastatic disease may give rise to nerve pain.

In one embodiment there is provided a use of an anti P2X₇ receptor antibody in the manufacture of a medicament for the treatment of neuropathic cancer pain in an individual arising from a primary tumour being a benign or malignant tumor, or from metastatic disease. Preferably the use results in the reduction of at least one symptom of the pain, preferably a symptom of local pain, preferably selected from the group consisting of burning, shooting or tingling pain.

In another embodiment there is provided an anti P2X₇ receptor antibody for use in the treatment of neuropathic cancer pain in an individual arising from a primary tumour being a benign or malignant tumor, or from metastatic disease. Preferably the use results in the reduction of at least one symptom of the pain, preferably a symptom of local pain, preferably selected from the group consisting of burning, shooting or tingling pain.

In another embodiment there is provided a method of managing neuropathic cancer pain in an individual including the following steps:
providing an individual in need of pain management arising from a primary tumour being a benign or malignant tumor, or from metastatic disease;
administering an anti-P2X₇ receptor antibody to the individual;

thereby managing pain in the individual. Preferably the method results in the reduction of at least one symptom of the pain, preferably a symptom of systemic or referred pain, preferably selected from the group consisting of burning, shooting or tingling pain.

In the treatment of neuropathic pain, an antibody that binds to functional and non functional P2X₇ receptors, or to non functional P2X₇ receptors only, or to functional P2X₇ receptors only may be used. In one embodiment the antibody binds to functional P2X₇ receptors. In another embodiment the antibody binds to non functional P2X₇ receptors only.

In another embodiment, the method, use of anti P2X₇ antibody for use in the treatment of pain may be co-administered with an anti cancer agent. In these embodiments, the anti cancer agent typically functions as a cytotoxic agent to kill cancer cells, thereby debulking the tumour mass, whether after surgery or other therapy, or as a front line procedure. Any anti cancer agent could be used, including a small molecule or a larger biological molecule such as an antibody. In these embodiments, the anti P2X₇ antibody plays an important role in reducing the production or formation of ATP in the cancer cells at the tumour margin which are exposed to the anti cancer agent before those cells are killed, thereby preventing or substantially preventing release of ATP that would otherwise activate pain receptors such as P2X₃.

A particularly useful anti-cancer agent is a cytotoxic antibody or antibody conjugate that is specific for an anti cancer cell antigen or biomarker, and in particular those biomarkers that have cancer specific expression.

Where the anti cancer agent is an anti cancer cell antigen antibody, it is particularly useful to administer the anti P2X₇ antibody and anti cancer cell antigen antibody simultaneously in the form of a multimeric antibody that includes at least a first variable domain for binding to a P2X₇ receptor and at least a second variable domain for binding to a cancer cell antigen, as this enables the second variable domain to assist in targeting the first variable domain to a cancer cell.

In other embodiments the anti P2X₇ antibody may be provided before an anti cancer agent is provided so as to enable the reduction of ATP production or expression by cancer cells at the tumour margin before they are lysed by a cytotoxic anti cancer agent.

### C. Minimisation and Management of Chronic Pain by Administration of P2X₇ Receptor or Fragments or Peptides Therefrom

In one embodiment of the invention, anti-P2X₇ antibodies are provided in the individual requiring pain management or minimisation by generating antibodies inside the individual. This can be achieved by forming a humoral immune response in the individual to an immunogen.

The immunogen may be provided in the form of a P2X₇ receptor, or a fragment of a P2X₇ receptor that is capable of inducing an immune response to a non-functional P2X₇ receptor in the individual. A non-functional P2X₇ receptor is defined as having at least one of the three ATP binding sites that are formed at the interface between adjacent correctly packed monomers that are unable to bind ATP. Such receptors are unable to extend the opening of the non-selective calcium channels to apoptotic pores.

The immunogen may contain at least one sequence that is capable of being presented on a major histocompatibility complex class II molecule and/or is capable of interacting with a T or B-cell receptor or a B-cell membrane bound-immunoglobulin.

Typically the immune response that is formed is specific for non-functional P2X₇ receptors, in which case antibodies or cellular components that are reactive with non-functional P2X₇ receptors (i.e. non ATP binding receptors), but not reactive with functional P2X₇ receptors (i.e. ATP binding receptors are) are formed in the individual.

In another embodiment there is provided a use of an anti-P2X₇ receptor, fragment thereof or peptide derived therefrom in the manufacture of a medicament for the treatment of pain in an individual.

In another embodiment there is provided an anti-P2X₇ receptor, fragment thereof or peptide derived therefrom for use in the treatment of pain in an individual.

In these embodiments, the anti-P2X₇ receptor, fragment thereof or peptide derived therefrom is utilised to form a humoral immune response to the receptor, fragment or peptide in the individual, thereby managing pain in the individual.

In one embodiment there is provided a method of managing pain in an individual including the following steps:
providing an individual in need of pain management;
administering an anti-P2X₇ receptor, fragment thereof or peptide derived therefrom to the individual thereby forming an humoral immune response to the receptor, fragment or peptide in the individual;

thereby managing pain in the individual.

In the above described embodiments, the individual may have chronic pain. The chronic pain is localised or systemic.

The pain may be cancer pain. Generally the invention is directed to the treatment of chronic cancer pain.

In one embodiment there is provided a use of an anti-P2X₇ receptor, fragment thereof or peptide derived therefrom, in the manufacture of a medicament for the treatment of deep somatic cancer pain in an individual arising from a primary tumour being a benign or malignant tumor, or from metastatic disease. Preferably the use results in the reduction of at least one symptom of the pain, preferably a symptom of systemic pain, preferably selected from the group consisting of localised throbbing, dull or aching pain.

In another embodiment there is provided an anti-P2X₇ receptor, fragment thereof or peptide derived therefrom, for use in the treatment of deep somatic cancer pain in an individual arising from a primary tumour being a benign or malignant tumor, or from metastatic disease. Preferably the use results in the reduction of at least one symptom of the pain, preferably a symptom of systemic pain, preferably selected from the group consisting of localised throbbing, dull or aching pain.

In another embodiment there is provided a method of managing deep somatic cancer pain in an individual including the following steps:
providing an individual in need of deep somatic cancer pain management arising from a primary tumour being a benign or malignant tumor, or from metastatic disease;
administering an anti-P2X₇ receptor, fragment thereof or peptide derived therefrom to the individual;

thereby managing pain in the individual. Preferably the method results in the reduction of at least one symptom of the pain, preferably a symptom of systemic pain, preferably selected from the group consisting of localised throbbing, dull or aching pain.

In one embodiment there is provided a use of an anti-P2X₇ receptor, fragment thereof or peptide derived therefrom in the manufacture of a medicament for the treatment of visceral cancer pain in an individual arising from a primary tumour being a benign or malignant tumor, or from metastatic disease. Preferably the use results in the reduction of at least one symptom of the pain, preferably a symptom of systemic or referred pain, preferably selected from the group consisting of pain arising from pressure or deep squeezing.

In another embodiment there is provided an anti-P2X₇ receptor, fragment thereof or peptide derived therefrom for use in the treatment of visceral cancer pain in an individual arising from a primary tumour being a benign or malignant tumor, or from metastatic disease. Preferably the use results in the reduction of at least one symptom of the pain, preferably a symptom of systemic or referred pain, preferably selected from the group consisting of pain arising from pressure or deep squeezing.

In another embodiment there is provided a method of managing visceral cancer pain in an individual including the following steps:
providing an individual in need of pain management arising from a primary tumour being a benign or malignant tumor, or from metastatic disease;
administering an anti-P2X₇ receptor, fragment thereof or peptide derived therefrom to the individual;

thereby managing pain in the individual. Preferably the method results in the reduction of at least one symptom of the pain, preferably a symptom of systemic or referred pain, preferably selected from the group consisting of pain arising from pressure or deep squeezing.

In one embodiment there is provided a use of an anti-P2X₇ receptor, fragment thereof or peptide derived therefrom in the manufacture of a medicament for the treatment of neuropathic cancer pain in an individual arising from a primary tumour being a benign or malignant tumor, or from metastatic disease. Preferably the use results in the reduction of at least one symptom of the pain, preferably a symptom of local pain, preferably selected from the group consisting of burning, shooting or tingling pain.

In another embodiment there is provided an anti-P2X₇ receptor, fragment thereof or peptide derived therefrom for use in the treatment of neuropathic cancer pain in an individual arising from a primary tumour being a benign or malignant tumor, or from metastatic disease.

Preferably the use results in the reduction of at least one symptom of the pain, preferably a symptom of local pain, preferably selected from the group consisting of burning, shooting or tingling pain.

In another embodiment there is provided a method of managing neuropathic cancer pain in an individual including the following steps:
providing an individual in need of pain management arising from a primary tumour being a benign or malignant tumor, or from metastatic disease;
administering an anti-P2X₇ receptor, fragment thereof or peptide derived therefrom to the individual;

thereby managing pain in the individual. Preferably the method results in the reduction of at least one symptom of the pain, preferably a symptom of systemic or referred pain, preferably selected from the group consisting of burning, shooting or tingling pain.

In another embodiment, the method, use of an anti-P2X₇ receptor, fragment thereof or peptide derived therefrom for use in the treatment of pain may be co-administered with an anti cancer agent. In these embodiments, the anti cancer agent typically functions as a cytotoxic agent to kill cancer cells, thereby debulking the tumour mass, whether after surgery or other therapy, or as a front line procedure. Any anti cancer agent could be used, including a small molecule or a larger biological molecule such as an antibody. In these embodiments, the anti P2X₇ antibody generated from the anti-P2X₇ receptor, fragment thereof or peptide derived therefrom plays an important role in reducing the production or formation of ATP in the cancer cells at the tumour margin which are exposed to the anti cancer agent before those cells are killed, thereby preventing or substantially preventing release of ATP that would otherwise activate pain receptors such as P2X₃.

A particularly useful anti-cancer agent is a cytotoxic antibody or antibody conjugate that is specific for an anti cancer cell antigen or biomarker, and in particular those biomarkers that have cancer specific expression.

In other embodiments the an anti-P2X₇ receptor, fragment thereof or peptide derived therefrom may be administered so as to produce an anti P2X₇ antibody in the individual before an anti cancer agent is provided so as to enable the reduction of ATP production or expression by cancer cells at the tumour margin before they are lysed by a cytotoxic anti cancer agent.

### D. Assessment, Categorisation and Monitoring of Symptoms of Chronic Cancer Pain

As discussed herein, symptoms of chronic cancer pain, especially somatic, visceral or neuropathic pain may manifest as aching, dull, throbbing, burning, shooting, tingling, sharp or cramping pain. The assessment and diagnosis of these symptoms is well known to the skilled worker. Further, techniques for monitoring the management of symptoms of chronic cancer pain are also well known to the skilled worker.

In embodiments the method may further include the step of:
assessing the individual to determine the level of reduction of symptoms of the pain in the individual after administration of the anti-P2X₇ receptor antibody, or after administration of the anti-P2X₇ receptor, fragment thereof or peptide derived therefrom;

thereby managing pain in the individual. Subsequent further administration and assessment steps may be implemented to manage pain in the individual. Thus in one embodiment the method may include the further steps of:
further administering an anti-P2X₇ receptor antibody or an anti-P2X₇ receptor, fragment thereof or peptide derived therefrom to the individual;
thereafter, further assessing the individual to determine the level of reduction of symptoms of the pain in the individual arising from said administration;

wherein said further administration and assessment are continued until the individual experiences a reduction in pain.

Typically, the individual is assessed to determine the level of reduction of a symptom of pain selected from the group consisting of: aching, dull, throbbing, burning, shooting, tingling, sharp or cramping pain.

Typically the management of pain in the individual results in the reduction of a symptom of pain selected from the group consisting of: aching, dull, throbbing, burning, shooting, tingling, sharp or cramping pain.

In one embodiment, the cancer pain is bone pain and the individual is assessed for reduction of symptoms of pain in the form of dull, aching or throbbing pain. The cancer pain may arise from the individual having sarcoma, osteosarcoma or metastatic disease and the management of the pain in the individual may result in the reduction of a symptom of pain in the form of dull, aching or throbbing pain.

In one embodiment, the cancer pain is visceral pain and the individual is assessed for reduction of symptoms of pain in the form of sharp, cramping, aching or throbbing. The cancer pain may arise from the individual having a primary tumour of an organ, or from metastatic disease and the management of pain in the individual may result in the reduction of a symptom of pain in the form of sharp, cramping, aching or throbbing.

In another embodiment the cancer pain is neuropathic pain and the individual is assessed for reduction of symptoms of pain in the form of burning, shooting or tingling pain symptoms. The cancer pain may arise from the individual having malignant or metastatic disease and the management of pain in the individual may result in the reduction of a symptom of pain in the form of burning, shooting or tingling symptoms.

In one embodiment, the individual selected for treatment is inappetent.

In another embodiment, the individual selected for treatment is a palliative care patient.

In one embodiment the individual selected for treatment has a Grade 1, 2, 3, or 4 tumour, preferably a Grade, 3 or 4 tumour, more preferably a Grade 4 tumour. In Grade 1 tumour, the tumour cells and the organization of the tumour tissue appear localized and close to normal with well differentiated cells. These tumours tend to grow and spread slowly. In contrast, the cells and tissue of Grade 3 (poorly differentiated) and Grade 4 (undifferentiated) tumours do not look like normal cells and tissue. Grade 3 and Grade 4 tumour tend to grow rapidly and spread faster than tumour with a lower grade.

### E. Anti P2X₇ Receptor Antibodies for Minimising Pain and Compositions Containing Same

As described herein, the invention is for the use of anti P2X₇ receptor antibodies for the management of pain, especially chronic cancer pain. These antibodies may be those that bind to non functional P2X₇ receptors, but not to functional P2X₇ receptors. Examples are disclosed in PCT/AU2002/000061, PCT/AU2002/001204, PCT/AU2007/001540, PCT/AU2007/001541, PCT/AU2008/001364, PCT/AU2008/001365, PCT/AU2009/000869 and PCT/AU2010/001070 the contents of which are incorporated by reference in their entirety.

In other embodiments, these antibodies may bind to an epitope that is common to functional P2X₇ receptors and non functional P2X₇ receptors. Examples of these epitopes include the E140 epitope, and generally epitopes found within the following regions of the P2X₇ receptor extracellular domain: 75-90; 120-130; 135-150; 310-320.

In other embodiments, these antibodies may bind to an epitope selected from the group consisting of E200, E300 and E200-300. “E200 epitope” generally refers to an epitope presented on a non-functional P2X₇ receptor. In humans the sequence is GHNYTTRNILPGLNITC (SEQ ID NO:2). “E300 epitope” generally refers to an epitope presented on a non-functional P2X₇ receptor. In humans the sequence is: KYYKENNVEKTLIKVF (SEQ ID NO:3). “Composite epitope” generally refers to an epitope that is formed from the juxtaposition of the E200 and E300 epitopes.

In a particularly preferred embodiment, the anti P2X₇ receptor antibody is 2-2-1 as described in PCT/AU2010/001070 the contents of which are incorporated by reference in their entirety.

The antibody may be provided in the form of an immunoglobulin variable domain, whole antibody, Fab, dab, scFv, diabody, triabody, fusion protein, conjugate, bispecific antibody or pharmaceutical composition

As described herein, the antibody may be monovalent, and in particular without a capacity for cross linking receptor on the cell surface, as is seen with bivalent and higher order valency antibodies. In one particular preferred embodiment, the antibody may be a single domain antibody.

As described herein, multimeric antibodies including at least one variable domain for binding to a P2X₇ receptor, enabling minimisation of pain, and a further variable domain for therapy of cancer may further be particularly useful for targeting of the P2X₇ receptor variable domain to a cancer cell. Such an antibody enables one to ostensibly turn off production or expression of ATP by cancer cells at the tumour margin thereby preventing ATP from being released when the tumour cell is lysed or otherwise killed by therapeutic antibody. Thus in one embodiment there is provided a multimeric antibody for minimising cancer pain including:
at least a first variable domain for binding to a P2X₇ receptor for minimising cancer pain
at least a second variable domain for binding to a cancer cell antigen.

Methods for producing multimeric antibodies are known to the skilled worker: See Powers G A, Hudson P J, Wheatcroft M P. 2012: Methods Mol Biol. 907:699-712. doi: 10.1007/978-1-61779-974-7 39. “Design and production of multimeric antibody fragments, focused on diabodies with enhanced clinical efficacy” the contents of which are incorporated by reference in their entirety.

Typically the anti P2X₇ antibodies for use in the invention bind to P2X₇ receptors on live cells with affinities (K_{D}) in the range of about 1 pM to about 1 uM. Typically, when the antibody is part of an IgM the affinity for P2X₇ receptors on live cells is between about 1 pM to about 1 nM, preferably about 1 pM to about 50 pM. Typically, when the antibody is part of an IgG the affinity for P2X₇ receptors on live cells is between about 1 pM to about 1 nM, preferably between about 1 pM to about 100 pM. Typically, when the antibody is part of a Fab the affinity for P2X₇ receptors on live cells is between about 100 pM to about 100 nM, preferably about 1 nM to about 100 nM. Typically, when the antibody is part of an scFV the affinity for P2X₇ receptors on live cells is between about 10 nM to about 1 uM, preferably about 10 nM to about 100 nM. Typically, when the antibody is part of a dAb the affinity for P2X₇ receptors on live cells is between about 10 nM to about 10 uM, preferably about 100 nM to about 1 uM.

### F. Dosage and Administration Regimes for Adoptive Transfer of Antibody

Typically the anti P2X₇ antibody is utilised in a pain alleviating amount. This amount may depend on the symptoms of pain that are experienced by the individual, by the type of cancer, by other anti cancer treatments or other pharmaceutical treatments and the general wellbeing of the individual. Treatment dosages may be titrated using routine methods known to those of skill in the art to optimize safety and efficacy.

In certain embodiments, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg kg, etc.), of the host body weight. For example dosages can be 1 mg/kg body weight or 10 mg/kg body weight or within the range of 1-60 mg/kg, preferably at least 1 mg/kg. Doses intermediate in the above ranges are also intended to be within the scope of the invention.

Subjects can be administered such doses daily, on alternative days, weekly or according to any other schedule determined by empirical analysis. An exemplary treatment entails administration in multiple dosages over a prolonged period, for example, of at least six months.

In one embodiment the anti P2X₇ antibody for use in the treatment of pain is administered for a period of no more than about 2 years.

In one embodiment where the anti P2X₇ antibody is 2-2-1, the antibody is given as a single dose in an amount of 5 mg/kg.

Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, more than one type of anti P2X₇ receptor antibody with different binding specificities may be administered simultaneously, in which case the dosage of each anti P2X₇ receptor antibody administered falls within the ranges indicated.

An anti-P2X₇ receptor antibody can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 ug/mL and in some methods 25-300 ug/mL.

In another embodiment, the anti P2X₇ antibody for use in the treatment of pain is administered at a frequency of 1 to 4 times per month.

Alternatively, anti-P2X₇ receptor antibody can be administered as a sustained release formulation, in which case less frequent administration is required. Dosage and frequency vary depending on the half-life of the antibody in the patient. The half-life of an antibody can also be prolonged via fusion to a stable polypeptide or moiety, e.g., albumin or PEG. In general, humanized antibodies show the longest half-life, followed by chimeric antibodies and nonhuman antibodies.

In one embodiment, the anti-P2X₇ receptor antibody can be administered in unconjugated form. In another embodiment the antigen binding sites for use in the methods disclosed herein can be administered multiple times in conjugated form. In still another embodiment, the antigen binding sites of the invention can be administered in unconjugated form, then in conjugated form, or vice versa.

As discussed herein, the mechanism for minimisation of chronic pain does not arise from antibody-induced killing of cancer cells. It follows that in certain embodiments the use of the antibody in the methods of the invention herein for pain minimisation or management do not result in substantial reduction in tumour mass over the time period in which pain treatment is required. Pain minimisation may be experienced 1 to 2 days after administration of antibody, establishing a period of minimised pain for 1 to 4 weeks after administration of the antibody. This dosing schedule is unlikely to result in significant debulking of tumour. Further, in these embodiments, the therapeutic or anti cancer effect is provided by another anti cancer drug as described herein.

### G. Dosage and Administration Regimes for Administration of P2X₇ Receptor or Fragments or Peptides Therefrom

The immunogen, in the form of P2X₇ receptor or fragments or peptides therefrom is provided in an initial administration to the individual, thereby forming a response that includes IgM production. In a further preferred form, the immunogen, which has been provided in an initial administration to the individual, thereby forming a response that includes IgM production, is administered at a later time, in a further administration to the initial administration, thereby forming a response that includes IgG production. In this embodiment, the further administration of immunogen may occur when the level of IgM in circulation in the individual is substantially undetectable.

The immune response formed is a humoral response, but may also have cellular components to the response, including cytotoxicity. A humoral response may include the transformation of B-cells into plasma cells that secrete antibody, Th2 activation and cytokine production, germinal centre formation and isotype switching, affinity maturation of B-cells and/or memory cell generation. A cellular response may include activating antigen-specific cytotoxic T-lymphocytes, activating macrophages and natural killer cells and/or stimulating cells to secrete cytokines. The humoral response may cause minimisation of pain by binding of the antibodies formed in the response to P2X₇ receptors on cancer cells.

Preferably a composition for use in these methods includes a carrier, excipient or diluent. Preferably, the composition further includes an adjuvant. In a preferred form, the composition enables the formation of a primary immune response (including IgM production) upon initial administration of the immunogen to the individual, and a second immune response (including IgG production) upon administration of the immunogen further to the initial administration.

In one embodiment the individual selected for treatment to minimise pain has not been treated with antibody immunotherapy or other form of therapy. In another embodiment, the individual selected for treatment according to a method described above is one who has received, or who are continuing to receive antibody immunotherapy, for treatment of cancer. Antibody immunotherapy generally means the administration of exogenous, (otherwise known as or “non self”) antibodies to an animal individual requiring treatment, as in the case of adoptive transfer of antibody. For example, the individual may have received any one of the therapeutic antibodies that have received regulatory approval for indications related to oncology.

The purpose of the treatment according to these methods of the invention is to at least minimise pain by induction or formation of an immune response in the individual to a P2X7 receptor. Therefore, the individual selected for treatment must be capable of generating an immune response sufficient for meeting this purpose. Generally the desired immune response includes a capacity to produce either or both of circulating IgM and IgG when the individual has active cancer or is challenged by cancer, as in recurrence of cancer.

Individuals having a capacity to generate the immune response described herein may be selected or screened by a variety of methods well known in the art for detection of immunodeficiency. Typically, the individual selected for treatment will be one having at least one white blood cell component count within normal parameters. For example, a human for inclusion is generally one having a white blood cell count of between 2-10×10⁹/L, or a lymphocyte count of between 0.5-5×10⁹/L. Neutrophil count may be between 1.5-7.5×10⁹/L; monocyte count 0.1-0.8×10⁹/L, eosinophil less than about 0.4×10⁹/L and basophil less than about 0.01×10⁹/L.

It will be understood that in certain embodiments the cell count for any one of these blood cell components may fall outside these stated ranges, particularly in circumstances where the individual has a form of blood cancer, for example CML, CLL etc.

Generally an important factor is the lymphocyte count and/or monocyte count. In more detail, where either or both of these counts are significantly below the stated ranges for these components, the individual may be less likely to respond to administration of the immunogen.

Generally the immunogen is one which elicits an immune response to non-functional P2X7 but not to functional P2X7 receptors.

The immunogen may include or consist of a peptide including a sequence of a P2X7 receptor. The peptide may contain at least one sequence that is capable of being presented on a major histocompatibility complex class II molecule or, that is capable of interacting with a B-cell receptor or a B-cell membrane bound-immunoglobulin. Typically the peptide includes a sequence of a human P2X7 receptor or fragment thereof.

A range of peptide immunogens are known and discussed in PCT/AU2002/000061, PCT/AU2002/000061, PCT/AU2008/001364 and PCT/AU2009/000869, the contents of which are incorporated in entirety. Exemplary peptides immunogens within these specifications which include epitopes for generating an immune response to a non-functional P2X7 receptor are described below.

| PCT application | Peptide immunogen sequence |
|---|---|
| PCT/AU2002/000061, | GHNYTTRNILPGLNITC |
| | (SEQ ID NO: 2) |
| PCT/AU2008/001364 | KYYKENNVEKRTLIKVF |
| | (SEQ ID NO: 3) |
| PCT/AU2009/000869 | GHNYTTRNILPGAGAKYYKENNVEK |
| | (SEQ ID NO: 4) |

It will be understood that these are merely examples of possible immunogens useful for forming an immune response. Further, the invention includes the use of other peptides as described in these applications useful for forming an immune response to non-functional P2X7 receptors.

Typically the immunisation regime involves 2 or more immunisations. In a first immunisation, the objective may be to develop an IgM response to immunisation. A second immunisation may be to develop and IgG response. Further immunisations may be to boost the IgG response.

Where the immunogen is a peptide, the peptide may be provided in an amount of about 0.01 to 1 mg per administration.

A further administration of about 0.3 mg peptide may be applied.

In one embodiment a first immunisation is performed and the level of IgM production is then monitored over the following weeks. At about 4 to 5 weeks after first immunisation, the level of IgM antibody is likely to have decreased to negligible circulating levels. At this point, a second immunisation is then performed and the level of IgG production is monitored over the following weeks. Further testing of immunity over the following months/years may be performed and boosting immunisations may be provided as required.

A peptide immunogen may have a length of 6, 7, 8, 9, 10, 11, 12, 13, 14, 15, 16, 17, 18, 19, 20, 21, 22, 23, 24, 25 or 26 residues.

In one embodiment, the immunogen for forming an immune response according to a method of the invention is a peptide having a sequence of a P2X7 receptor that may or may not have Pro210 in cis conformation.

The immunogen may be in the form of a P2X7 extracellular domain or any one or more of the P2X7 isoforms. The immunogen may be provided for administration in a soluble form or associated with a solid phase such as a cell membrane, bead, or other surface.

Methods for screening peptides that can be used as an immunogen to form an immune response according to the methods of the invention herein are disclosed herein. One example includes the use of erythrocytes in a rosetting assay. In this assay an antibody that binds to functional receptors is used as a positive control in which rossettes are observed. A test antibody is determined not to bind to functional receptors if it fails to form rossettes. It is determined to bind to non-functional receptors if it is observed to bind to a non-functional receptor-expressing cell line, including those discussed herein.

The peptides of the invention can be made by any number of techniques known in the art including solid phase synthesis and recombinant DNA technology.

As is known in the art, a carrier is a substance that may be conjugated to a peptide epitope thereby enhancing immunogenicity. Some carriers do this by binding to multiple peptides so as to provide an antigen of increased molecular weight to the host in which the immune response is to be developed.

Preferred carriers include bacterial toxins or toxoids. Other suitable carriers include the N. meningitides outer membrane protein, albumin such as bovine serum albumin, synthetic peptides, heat shock proteins, KLH, Pertussis proteins, protein D from H. influenza and toxin A, B or C from C. difficile.

When the carrier is a bacterial toxin or toxoid, diphtheria or tetanus toxoids are preferred.

Preferably the carrier contains functional groups that can react with the peptide of the invention, or may be modified to be capable of reacting with the peptide.

The immunogen may be administered subcutaneously, intradermally and/or intramuscularly.

In a preferred form, the composition for forming an immune response to a P2X7 receptor for use in the methods of the invention described herein includes an adjuvant or compound for potentiating an immune response.

A large number of adjuvants are known; See also Allison (1998, Dev. Biol. Stand., 92:3-11; incorporated herein by reference), Unkeless et al. (1998, Annu. Rev. Immunol., 6:251-281), and Phillips et al. (1992, Vaccine, 10:151-158). Exemplary adjuvants that can be utilized in accordance with the invention include, but are not limited to, cytokines, aluminium salts (e.g., aluminium hydroxide, aluminium phosphate, etc.; Baylor et al., Vaccine, 20:S18, 2002), gel-type adjuvants (e.g., calcium phosphate, etc.); microbial adjuvants (e.g., immunomodulatory DNA sequences that include CpG motifs; endotoxins such as monophosphoryl lipid A (Ribi et al., 1986, Immunology and Immunopharmacology of bacterial endotoxins, Plenum Publ. Corp., NY, p 407, 1986); exotoxins such as cholera toxin, E. coli heat labile toxin, and pertussis toxin; muramyl dipeptide, etc.); oil-emulsion and emulsifier-based adjuvants (e.g., Freund's Adjuvant, MF59 [Novartis], SAF, etc.); particulate adjuvants (e.g., liposomes, biodegradable microspheres, etc.); synthetic adjuvants (e.g., nonionic block copolymers, muramyl peptide analogues, polyphosphazene, synthetic polynucleotides, etc.); and/or combinations thereof. Other exemplary adjuvants include some polymers (e.g., polyphosphazenes; described in U.S. Pat. No. 5,500,161), Q57, saponins (e.g., QS21, Ghochikyan et al., Vaccine, 24:2275, 2006), squalene, tetrachlorodecaoxide, CPG 7909 (Cooper et al., Vaccine, 22:3136, 2004), poly[di(carboxylatophenoxy)phosphazene] (PCCP; Payne et al., Vaccine, 16:92, 1998), interferon-γ (Cao et al., Vaccine, 10:238, 1992), block copolymer P1205 (CRL1005; Katz et al., Vaccine, 18:2177, 2000), interleukin-2 (IL-2; Mbwuike et al., Vaccine, 8:347, 1990), polymethyl methacrylate (PMMA; Kreuter et al., J. Pharm. ScL, 70:367, 1981), etc.

In one embodiment, a peptide immunogen containing a sequence of a P2X7 receptor is provided on the surface of a bacteriophage for immunisation of an animal according to a method of the invention described herein.

An individual requiring pain management or minimisation according to the invention is typically a mammal, and preferably a human although other non human mammals include companion animals and performance animals.

### Example 1

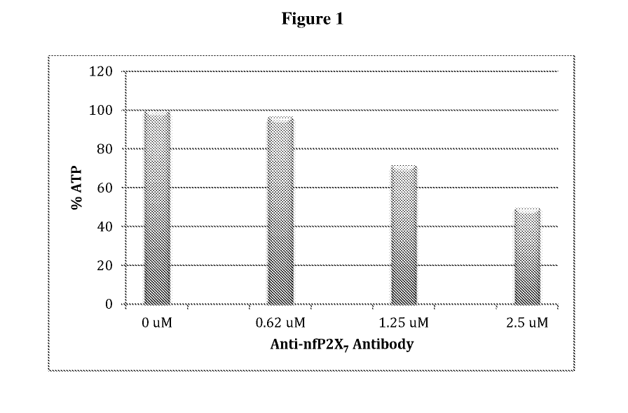

The extracellular ATP detected in the supernatant and intracellular stores of growing tumour cells in culture was measured using the Promega ATP Lite Detection System. Changes in the detected levels followed incubation of COLO205 cells with specific antibodies bound to the surface nfP2X₇ receptors. FIG. 1 shows that incubation with increasing concentrations of a specific IgG antibody labelled with FITC causes progressive diminution of the total ATP in the culture over a 5 h incubation period (50,000 cells/well in triplicate).

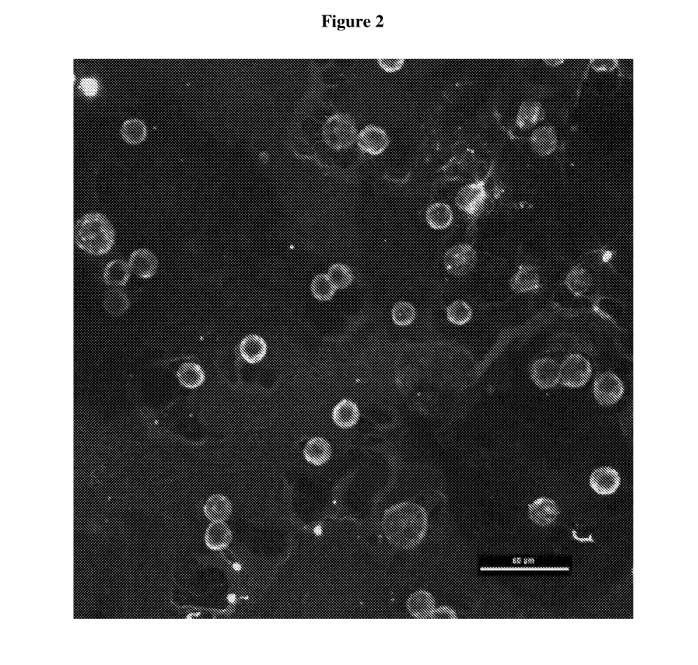

Such a short incubation period was not associated with cell death. Endocytosis of the labelled antibody at 37 C was apparent. Over half the cells in culture showed a heavy cytoplasmic distribution of the FITC label as shown in the confocal image in FIG. 2.

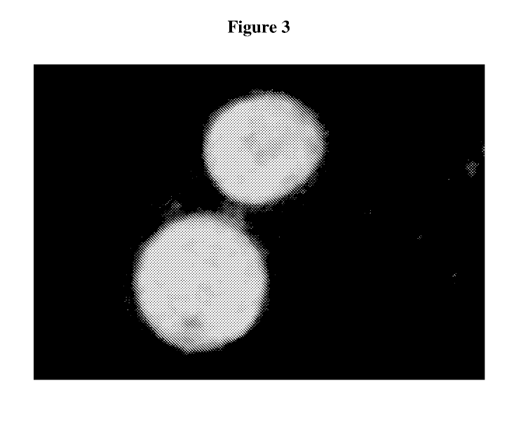

Some COLO205 cells showed intense internalisation after 3 h at 37 C as shown in FIG. 3 while others showed little effect.

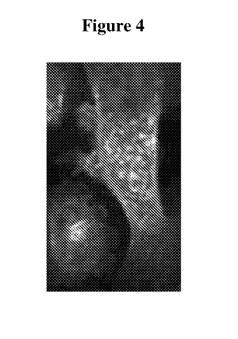

Similarly, prostate PC3 cells endocytosed the specific FITC-labeled anti-nfP2X₇ antibody (type IgG) in 2 h (FIG. 4). The antibody is seen in the EEA1-labeled endosomes in the cytoplasm beneath the phalloidin-stained actin situated under the plasma membrane. Nuclei are stained blue.

### Example 2

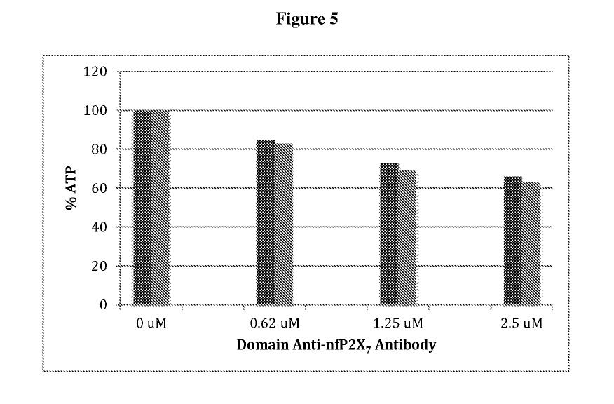

The issue of whether target receptor cross-linking is required to elicit inhibition of release of ATP was addressed by comparing the effects of a single headed domain antibody of type V_{H} with a DID-labelled Ab. As FIG. 5 shows, very similar ATP inhibition was elicited at the same concentrations of two-headed (left hand bars, blue) and single domains (right hand bars, red).

### Example 3

Since the amount of measurable ATP in supernatant is only of order 1% of the total intracellular stores, the inhibition of ATP is best measured after cell lysis. ATP released into the supernatant is hydrolysed rapidly. The progressive loss of total ATP follows the endocytosis of the receptors bound with anti-P2X₇ antibody, where the format is either domain, di-domain or full antibody. ATP stores are depleted in concert with the energy supplied via the ATP used to drive the endocytosis.

The total of ATP available to be released by growing tumour cells is thus reduced in the presence of saturating levels of bound antibody of various formats reducing the supply of agonist available for activating the purinergic pain pathway, utilising receptors such as P2X₃ (Chessell I P, Hatcher J P, Bountra C, Michel A D, Hughes J P, Green P, Egerton J, Muffin M, Richardson J, Peck W L, Grahames C B, Casula M A, Yiangou Y, Birch R, Anand P and Buell G N. Disruption of the P2X7 purinoceptor gene abolishes chronic inflammatory and neuropathic pain. Pain 2005, 114: 386-96).

The relief from systemic pain signals triggered by the agonist supplied by tumour cells is expected to occur over 1-2 days in the case of an adequate dose of exogenous antibody. Higher affinity formats applied via IV may well use much lower doses to achieve the same effect. This may include higher affinity antibodies or higher avidities achieved via multivalent presentation in which the dose may be reduced below 1 mg/kg.

### Example 4

A 3 yo male neutered cat weighing 3.7 kg was brought to the veterinary oncology clinic to be euthanased six weeks after a 1 cm diameter pancreatic cancer primary was detected. In the intervening period the primary had grown to 3 cm, had metastasised to liver (4 cm diameter secondary) and omentum (numerous secondaries up to 1 cm diameter). The patient was no longer able to stand nor eat, these symptoms being hallmarks of an individual suffering from systemic nociceptive pain. An infusion of sheep-derived P2X7-specific antibody was arranged with 2 mg/kg administered IP and 2 mg/kg administered IV via a catheter in the leg applied over 20 minutes (volume 5 mL). Within 2 days the patient regained appetite and demeanour that included constant purring and playing, suggesting a significant reduction in pain. He showed no gastrointestinal issues. The cats behaviour and demeanour suggests that this significant reduction in pain was maintained with the weekly treatments.

### Example 5

A 33.6 kg 13 yo female Golden Retriever presented to the clinic as a referral client with severe left forelimb lameness. She was unable to stand. CT (contrast and plain) showed marked lysis of left proximal humerus. She was receiving a non-steroidal anti-inflammatory, tramadol and gabapentin for pain relief.

A 10 mg/kg IV delivery of sheep-derived P2X7 antibody was administered over a 4 h period in 500 mL of Hartmann's solution after being premedicated with an antihistamine as a precaution against anti-sheep antibody effects. There was no adverse reaction to the infusion. In the subsequent days the family reported that she quickly regained her appetite and showed “obvious signs of contentment”. They reported that her mobility had improved as shown by an increased willingness to use the stairs, wanting to go for a walk, following family members around. Pain was no longer an obvious feature.

Treatment continued weekly for 6 weeks.

### Example 6

An example was a 42 yo male with end-stage oesophageal cancer that had metastasised to the liver and peritoneal wall. In the weeks prior to treatment, he had lost approximately 30 kg and had the hallmarks of significant nociceptive pain, treated with a combination of pain medication and was experiencing sleep deprivation. After the initial infusion of 5 mg/kg of human specific antibody administered over 2 h, he experienced a reduction in pain, no longer requiring breakthrough doses of medication, his sleeping initially improved, he became clearer and he was able to hold a coherent conversation. Prior to his second treatment, he had lost a further approximately 10 kg and was unconscious on the morning of the treatment. After the second treatment, he was coherent, ate his first meal in months, had a reduction in pain and a corresponding reduction in his pain management regime. Prior to his third treatment, his pain returned requiring increased pain management and palliative care involvement. After his third treatment, he became more coherent.

### Example 7

An example was an end-stage 54 yo patient with large volume systemic metastatic disease originating from a basal cell carcinoma. Treatment was administered in the form of a peptide therapeutic vaccine designed to generate an endogenous specific antibody response to the nfP2X7 receptor expression. The patient was initially ambulatory but weak and in significant discomfort. The initial treatment was applied subcutaneously and boosts were applied at 2 week intervals, this being a dosage regime designed for formation of humoral responses and from which humoral responses have been detected in other experiments. The patient's overall quality of life improved as evidenced by pain mitigation and improvements to the patients skin tone, general energy and overall demeanour.

It will be understood that the invention disclosed and defined in this specification extends to all alternative combinations of two or more of the individual features mentioned or evident from the text or drawings. All of these different combinations constitute various alternative aspects of the invention.

1. A method of managing pain in an individual including the following steps:
providing an individual in need of pain management;
administering an anti-P2X₇ receptor antibody to the individual;
thereby managing pain in the individual.

2. The method of claim 1 further including the step of:
assessing the individual to determine the level of reduction of symptoms of the pain in the individual after administration of the anti-P2X₇ receptor antibody;
thereby managing pain in the individual.

3. The method of claim 2 including the further steps of:
further administering an anti-P2X₇ receptor antibody to the individual;
thereafter, further assessing the individual to determine the level of reduction of symptoms of the pain in the individual arising from said administration;
wherein said further administration and assessment are continued until the individual experiences a reduction in pain.

4. The method of claim 1 wherein the individual has chronic pain or cancer pain.

5. (canceled)

6. (canceled)

7. The method of claim 1 wherein the cancer pain is selected from the group consisting of: nociceptive pain and neuropathic pain.

8. The method of claim 7 wherein the individual does not have neuropathic pain, or pain arising from nerve or nervous tissue injury.

9. (canceled)

10. (canceled)

11. The method of claim 6 wherein the individual is assessed to determine the level of reduction of a symptom of pain selected from the group consisting of: aching, dull, throbbing, burning, shooting, tingling, sharp or cramping pain.

12. (canceled)

13. The method of claim 1 wherein the cancer pain is bone pain and the individual is assessed for reduction of symptoms of pain in the form of dull, aching or throbbing pain.

14. (canceled)

15. The method of claim 13 wherein the management of pain in the individual results in the reduction of a symptom of pain in the form of dull, aching or throbbing pain.

16. The method of claim 1 wherein the cancer pain is visceral pain and the individual is assessed for reduction of symptoms of systemic or referred pain in the form of pain arising from pressure or deep squeezing or pain in the form of sharp, cramping, aching or throbbing pain symptoms.

17. The method of claim 16 wherein the cancer pain arises from the individual having a primary tumour of an organ, or from metastatic disease.

18. The method of claim 16 wherein the management of pain in the individual results in the reduction of a symptom of systemic or referred pain in the form of pain arising from pressure or deep squeezing or pain in the form of sharp, cramping, aching or throbbing pain symptoms.

19. The method of claim 1 wherein the cancer pain is neuropathic pain and the individual is assessed for reduction of symptoms of pain in the form of burning, shooting or tingling pain symptoms.

20. The method of claim 19 wherein the cancer pain arises from the individual having malignant or metastatic disease.

21. The method of claim 19 wherein the management of pain in the individual results in the reduction of a symptom of pain in the form of burning, shooting or tingling pain symptoms.

22. The method of claim 1 wherein the antibody binds to non-functional P2X₇ receptors but not to functional P2X₇ receptors.

23. A method of managing pain in an individual including the following steps:
providing an individual in need of pain management;
administering an anti-P2X₇ receptor, fragment thereof or peptide derived therefrom to the individual thereby forming an humoral immune response to the receptor, fragment or peptide in the individual;
thereby managing pain in the individual.

24. The method of claim 23 further including the step of:
assessing the individual to determine the level of reduction of symptoms of the pain in the individual after administration of the anti-P2X₇ receptor, fragment thereof or peptide derived therefrom;
thereby managing pain in the individual.

25. The method of claim 24 including the further steps of:
further administering an anti-P2X₇ receptor, fragment thereof or peptide derived therefrom to the individual;
thereafter, further assessing the individual to determine the level of reduction of symptoms of the pain in the individual arising from said administration;
wherein said further administration and assessment are continued until the individual experiences a reduction in pain.

26. The method of claim 25 wherein the individual has chronic pain or cancer pain.

27. (canceled)

28. (canceled)

29. The method of claim 28 wherein the cancer pain is selected from the group consisting of: nociceptive pain and neuropathic pain.

30. (canceled)

31. (canceled)

32. The method of claim 28 wherein the individual is assessed to determine the level of reduction of a symptom of pain selected from the group consisting of: aching, dull, throbbing, burning, shooting, tingling, sharp or cramping pain.

33. (canceled)

34. The method of claim 23 wherein the cancer pain is bone pain and the individual is assessed for reduction of symptoms of pain in the form of dull, aching or throbbing pain.

35. The method of claim 34 wherein the cancer pain arises from the individual having sarcoma, osteosarcoma or metastatic disease.

36. The method of claim 34 wherein the management of pain in the individual results in the reduction of a symptom of pain in the form of dull, aching or throbbing pain.

37. The method of claim 23 wherein the cancer pain is visceral pain and the individual is assessed for reduction of symptoms of systemic or referred pain in the form of pain arising from pressure or deep squeezing or pain in the form of sharp, cramping, aching or throbbing pain symptoms.

38. The method of claim 37 wherein the cancer pain arises from the individual having a primary tumour of an organ, or from metastatic disease.

39. The method of claim 37 wherein the management of pain in the individual results in the reduction of a symptom of systemic or referred pain in the form of pain arising from pressure or deep squeezing or pain in the form of sharp, cramping, aching or throbbing pain symptoms.

40. The method of claim 23 wherein the cancer pain is neuropathic pain and the individual is assessed for reduction of symptoms of pain in the form of burning, shooting or tingling pain symptoms.

41. The method of claim 40 wherein the cancer pain arises from the individual having malignant or metastatic disease.

42. The method of claim 40 wherein the management of pain in the individual results in the reduction of a symptom of pain in the form of burning, shooting or tingling pain symptoms.

43. (canceled)


SEQUENCE LISTING (4 sequences)

SEQ ID NO: 1 - protein, 595 aa, Homo sapiens [sequence omitted]

SEQ ID NO: 2 - protein, 17 aa, Homo sapiens
GHNYTTRNILPGLNITC

SEQ ID NO: 3 - protein, 17 aa, Homo sapiens
KYYKENNVEKRTLIKVF

SEQ ID NO: 4 - protein, 25 aa, Homo sapiens
GHNYTTRNILPGAGAKYYKENNVEK